(19) United States
(12) Patent Application Publication
(10) Pub. No.: US 2013/0338756 A1
(43) Pub. Date: Dec. 19, 2013

(54) STENT COMPOSED OF AN IRON ALLOY

(71) Applicant: Biotronik AG, Buelach (CH)

(72) Inventors: Bodo Gerold, Karlstadt (DE); Heinz Mueller, Erlangen (DE); Peter Uggowitzer, Ottenbach (CH)

(73) Assignee: Biotronik AG, Buelach (CH)

(21) Appl. No.: 13/898,633

(22) Filed: May 21, 2013

Related U.S. Application Data
(60) Provisional application No. 61/660,818, filed on Jun. 18, 2012.

(51) Int. Cl. A61F 2/06

(52) U.S. Cl. CPC A61F 2/06
USPC 623/1.15

(57) ABSTRACT

Some embodiments of the invention relate to a stent which is composed entirely or in parts of an iron alloy having the following composition (in % by weight): Cr: >12.0; Ni: 0-8.0; Co: 0-20.0; Mn: 0-20.0; N: 0.05-1.0; C 0.05-0.4; Ti: 0-3.5; Nb: 0-3.5; V: 0-3.5; Mo 0-3.5; Si: 0-3.0; Al: 0-3.0; and Cu: 0-3.0. A cumulative content of Co and Mn is 3.0-20.0% by weight. Iron and production-related impurities make up the remainder of the 100% by weight.

### CROSS REFERENCE The present application claims priority on copending U.S. Provisional Application No. 61/660,818 filed on Jun. 18, 2012; which application is incorporated herein by reference.

### TECHNICAL FIELD

Some embodiments of the invention relate generally to a stent which is composed entirely or in parts of an iron alloy.

### BACKGROUND

Stent implantation has become established as one of the most effective therapeutic measures for treating vascular disease. Stents are used to provide support in a patient's hollow organs. For this purpose, stents of a conventional design have a filigree support structure composed of metallic struts; the support structure is initially provided in a compressed form for insertion into the body, and is expanded at the application site. One of the main applications of stents of this type is to permanently or temporarily widen and hold open vasoconstrictions, in particular constrictions (stenoses) of the coronary arteries. In addition, aneurysm stents are known, for example, which are used primarily to seal the aneurysm.

Stents include a circumferential wall having a support force that suffices to hold the constricted vessel open to the desired extent; stents also include a tubular base body through which blood continues to flow without restriction. The circumferential wall is typically formed by a latticed support structure that enables the stent to be inserted, in a compressed state having a small outer diameter, until it reaches the constriction in the particular vessel to be treated, and to be expanded there, e.g. using a balloon catheter, until the vessel finally has the desired, enlarged inner diameter. Alternatively, materials having a memory effect, such as Nitinol, are capable of self-expansion in the absence of a restoring force that holds the implant at a small diameter. The restoring force is typically exerted on the material by a protective tube.

The stent comprises a base body made of an implant material. An implant material is a nonliving material that is used for a medical application and interacts with biological systems. A prerequisite for the use of a material as an implant material that comes in contact with the physical surroundings when used as intended is its biocompatibility. “Biocompatibility” refers to the capability of a material to evoke an appropriate tissue response in a specific application. This includes an adaptation of the chemical, physical, biological, and morphological surface properties of an implant to the recipient tissue, with the objective of achieving a clinically desired interaction. The biocompatibility of the implant material is furthermore dependent on the timing of the response of the biosystem in which the implant is placed. For example, irritations and inflammations, which can cause tissue changes, occur over the relatively short term. Biological systems therefore respond differently depending on the properties of the implant material. Depending on the response of the biosystem, implant materials can be subdivided into bioactive, bioinert, and degradable/resorbable (referred to here as biocorrodible) materials.

Implant materials include polymers, metallic materials, and ceramic materials (as a coating, for example). Biocompatible metals and metal alloys for permanent implants contain e.g. stainless steels (e.g. 316L), cobalt-based alloys (e.g. CoCrMo casting alloys, CoCrMo forging alloys, CoCrWNi forging alloys, and CoCrNiMo forging alloys), pure titanium and titanium alloys (e.g. CP titanium, TiAl6V4 or TiAl6Nb7), and gold alloys. In the field of biocorrodible stents, the use of magnesium or pure iron and biocorrodible base alloys of the elements magnesium, iron, zinc, molybdenum, and tungsten is proposed.

### SUMMARY

Stents of the invention are capable of withstanding great plastic elongation and of retaining their size and diameter after expansion. Basically, at least some stents of the invention:
have a small profile; this includes the suitability for crimping onto a balloon catheter.
have good expansion behavior; when the stent is inserted into the lesion and the balloon is expanded, the stent should expand uniformly in order to conform to the vessel wall.
have adequate radial strength and negligible recoil; once the stent has been placed, it should withstand the restoring forces of the vessel wall and not collapse.
have adequate flexibility, thereby enabling the stent to be conveyed through vessels and stenoses having a small diameter.
have appropriate x-ray visibility and MRI compatibility, thereby enabling the physician to evaluate the implantation and position of the stent in vivo.
have low thrombogenicity; the material should be biocompatible and, in particular, prevent the deposition and clumping of blood platelets.
be capable of releasing active agent; this prevents restenosis in particular.

The features apply in particular to the mechanical properties of the material of which the stent is produced. It is favorable to have high yield strength (the load at which plastic deformation of the material begins) combined with high maximum strength. The ratio of yield strength/maximum strength (yield ratio) should be as low as possible, since otherwise an increasingly greater portion of deformation occurs elastically, thereby resulting in high elastic recoil.

The materials 316L (Fe-base alloy), MP35N and L-605 (Co-base alloys), which are used to construct balloon-expandable stents, already have high strength and high fracture strain, but exhibit limits specifically in attempts to optimize the above-noted properties (simultaneous improvement of strength, yield strength, and yield ratio). This limits freedom in stent design development and use of the prior art:
(i) to low (tensile) strength Rm (UTS) and plastic elongation At (elongation at fracture)
As a result, the collapse pressure or radial strength is lower, and therefore thicker stent struts are required to prevent a reduction of the lumen or a stent collapse caused by the forces of elastic relaxation of the expanded vessel. The crimp profile is therefore thicker, which results in a greater reduction in the lumen, thereby delaying healing (endothelization) in the vessel wall.
   (ii) the (tensile) strength can be increased only if a smaller fracture strain or an overproportional increase in the yield strength Rp0.2 (YTS) can be accepted simultaneously. However, this results in an increased tendency of the stent to fracture, or in greater recoil after expansion. Strong elastic recoil results in a reduction in lumen after implantation, and in poorer crimpability, thereby increasing the risk of the stent becoming detached from the catheter.

As a result, demand persists for a metallic implant material that is suited for the production of stents. Embodiments of the invention have been discovered to address these otherwise unsatisfied needs.

### DETAILED DESCRIPTION

Embodiments of the invention include stents made of a novel alloy. Various elements of stent embodiments of the invention are known in the art and need not be illustrated herein for purposes of brevity. These elements include, for example, a generally tubular base body through which blood can flow without restriction, a generally latticed support structure of struts configured for stent insertion in a compressed state until reaching desired location where it is then expanded (using, for example, a balloon catheter removably held in the base body interior), one or more coatings on the all or a portion of the base body, and the like. Such features have been described in the background and are also readily known in the art.

The stent according to the present disclosure solves or ameliorates one or more of the above-described problems. At least some stent embodiments are composed entirely of, while other embodiments are composed at least partially of, an iron alloy having the composition:
Cr: >12.0% by weight
Ni: 0-8.0% by weight
Co: 0-20.0% by weight
Mn: 0-20.0% by weight
N: 0.05-1.0% by weight
C: 0.05-0.4% by weight
Ti: 0-3.5% by weight
Nb: 0-3.5% by weight
V: 0-3.5% by weight
Mo: 0-3.5% by weight
Si: 0-3.0% by weight
Al: 0-3.0% by weight
Cu: 0-3.0% by weight
wherein a cumulative content of Co and Mn is 3.0-20.0% by weight, and iron and production-related impurities make up the remainder of the 100% by weight, and
(i) a Cr-eq value for Cr equivalents that results from the % by weight portions of the stated alloy components represented by formula (1)

Cr-eq=[Cr]+1.5×[Mo]+0.48×[Si]+2.5×[Al]+1.75×[Nb]+2.3×[V] (1)
is greater than 18;
(ii) a Ni-eq value for Ni equivalents that results from the % by weight portions of the stated alloy components represented by formula (2)

Ni-eq=[Ni]+[Co]+30×[C]+18×[N]+0.1×[Mn]−0.01×[Mn]² (2)
is less than 22;
(iii) a PRE value for corrosion resistance that results from the % by weight portions of the stated alloy components represented by formula (3)

PRE=[Cr]+3.3×[Mo]+20×[N] (3)
is greater than 25;
(iv) the limitation represented by formula (4) applies for the Cr-eq and Ni-eq values

Ni-eq>Cr-eq−8 (4)
and
(v) formulas (5) and (6) apply for the limitations on the content of nitrogen and carbon

0.25≦C+N≦1.00 (5)

0.25≦C/N≦1.00 (6)

The alloy may be used, for example, to construct some or all of a stent base body (including struts and any other components thereof), which may be generally tubular shaped and include a lattice of expandable struts that define the tubular side walls. Stents of the invention may also include one or more coatings on all or part of the base body. The coatings may be useful to, among other things, reduce corrosion and to carry a drug for release in the body.

Other alloy concentrations will also be useful in alternative invention embodiments. As an example, some other stent embodiments are composed entirely or in parts of an iron alloy having the composition as outlined above, where the concentrations of one or more of, and in some embodiments each of, Ni, Co, Mn, Ti, Nb, V, Mo, Si, Al, and Cu are at least 0.05% by weight.

Alloys useful in invention embodiments may also include additional metal components.

It has been discovered that the Fe base alloys used according to the present disclosure are resistant to corrosion and frictional wear, and have a high cold-deformation capacity, excellent viscosity properties, and high strength. A portion of austenite in the alloy is preferably greater than 95% (i.e., more than 95% of Fe is austenite); and in some embodiments the alloy is present entirely in austenitic modification. The CrMnNi steel exhibits transformation-induced plasticity (TRIP) effects and twinning-induced plasticity (TWIP) effects. Alloy components Co, Mn, and N stabilize the austenitic state. In addition, Si, Al and Cu are added as alloy components that increase stacking fault energy.

The alloys used according to at least some embodiments of the invention have a very high strength Rm of >800 MPa, preferably >900 MPa. It has been discovered that the high strength makes it possible to attain thin structures in the stent design that nevertheless provide the stent with a high radial strength of >1.5 bar (150 kPa).

The alloys according to the at least some embodiments of also exhibit excellent deformability at room temperature. The degree of deformation (fracture strain) A is >40%, preferably >60%.

The alloys according to the at last some embodiments of invention have high resistance to local corrosion, i.e. pitting. This resistance can be specified by assigning the stated pitting resistance equivalent (PRE) value. PRE is preferably greater than 18, in some embodiments is greater than 28, in some embodiments is 30, and in some embodiments is greater than 30.

Cr-eq is greater than 18, preferably greater than 20, and Ni-eq is less than 22, preferably less than 18. It has been discovered that the inequality of formula (4) ensures that work is always performed in the austenitic range, i.e. no ferrite is present, and therefore ferromagnetism can be avoided. If a high PRE is desired, then Cr-eq and Ni-eq are likewise high.

Alloys useful in invention embodiments can be produced in a manner analogous to the usual production methods for iron-base alloys.

Invention embodiments are not limited to stents, but may include other implants as well. Additionally, invention embodiments include methods of making a stent or other implant including steps of using an alloy of the invention to form the stent or implant. Various steps of such formation are generally known in the art and need not be discussed in detail herein.

### EMBODIMENT 1

A Ni-free alloy having the composition (in % by weight) 17% Cr, 0.5% Mo, 10% Mn, 2% Si, 0.25% C and 0.4% N was melted in a vacuum melting furnace in a nitrogen atmosphere with a partial pressure of approximately 1 bar, and was cast into bars 8 cm×8 cm in size. After deformation by forging to form rods 2.5 cm×2.5 cm in size, they were solution-annealed for 6 h at a temperature of 1,150° C. and quenched in water. The material exhibits a homogeneous microstructure having a particle size of approximately 20 μm. The following characteristic values apply for alloys manufactured in this manner:

Cr-eq: 18.7; Ni-eq: 16.5; PRE: 26.7

Rp0.2=540 MPa; Rm=920 MPa; A=65%

### EMBODIMENT 2

An alloy having the composition (in % by weight) 17% Cr, 1.5Mo, 5.5% Ni, 7% Mn, 2% Si, 0.1% C and 0.25% N was melted in a vacuum melting furnace in a nitrogen atmosphere with a partial pressure of approximately 1 bar, and was cast into bars 8 cm×8 cm in size. After deformation by forging to form rods 2.5 cm×2.5 cm in size, they were solution-annealed for 6 h at a temperature of 1,150° C. and quenched in water. The material exhibits a homogeneous microstructure having a particle size of approximately 25 μm. The following characteristic values apply for alloys manufactured in this manner:

Cr-eq: 20.2; Ni-eq: 13.2; PRE: 26.0

Rp0.2=405 MPa; Rm=890 MPa; A=70%

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this present disclosure.

What is claimed is:

1. A stent composed at least partially of an iron alloy having the composition:
Cr greater than 12.0% by weight;
Ni between 0 and 8.0% by weight;
Co between 0 and 20.0% by weight;
Mn between 0 and 20.0% by weight;
N between 0.05 and 1.0% by weight;
C between 0.05 and 0.4% by weight;
Ti between 0 and 3.5% by weight;
Nb between 0 and 3.5% by weight;
V between 0 and 3.5% by weight;
Mo between 0 and 3.5% by weight;
Si between 0 and 3.0% by weight;
Al between 0 and 3.0% by weight; and
Cu between 0 and 3.0% by weight,
wherein a cumulative content of Co and Mn is between 3.0 and 20.0% by weight, and iron and production-related impurities make up a remainder of 100% by weight, and
(i) a Cr-eq value for Cr equivalents that results from the % by weight portions of the stated alloy components represented by formula (1)
Cr-eq=[Cr]+1.5×[Mo]+0.48×[Si]+2.5×[Al]+1.75×[Nb]+2.3×[V] (1)
is greater than 18;
(ii) a Ni-eq value for Ni equivalents that results from the % by weight portions of the stated alloy components represented by formula (2)
Ni-eq=[Ni]+[Co]+30×[C]+18×[N]+0.1×[Mn]−0.01×[Mn]² (2)
is less than 22;
(iii) a PRE value for corrosion resistance that results from the % by weight portions of the stated alloy components represented by formula (3)
PRE=[Cr]+3.3×[Mo]+20×[N] (3)
is greater than 25;
(iv) the limitation represented by formula (4) applies for the Cr-eq and Ni-eq values
Ni-eq>Cr-eq−8 (4);
and
(v) formulas (5) and (6) apply for the limitations on the content of nitrogen and carbon
0.25≦C+N≦1.00 (5),
0.25≦C/N≦1.00 (6).

2. The stent according to claim 1, wherein the Cr-eq value is greater than 20.

3. The stent according to claim 1, wherein the Ni-eq value is less than 18.

4. The stent according to claim 1, wherein the PRE value is greater than 28.

5. The stent according to claim 1, wherein the alloy has strength Rm that is greater than 800 MPa.

6. The stent according to claim 1, wherein the alloy has strength Rm that is greater than 900 MPa.

7. The stent according to claim 1, wherein the alloy has radial strength of greater than 1.5 bar.

8. The stent according to claim 1 wherein the stent has a basic body that is composed entirely of the alloy.

9. The stent according to claim 1 wherein the concentration of each of Ni, Co, Mn, Ti, Nb, V, Mo, Si, Al and Cu is at least 0.05% by weight.

10. The stent according to claim 1 wherein the portion of austenite in the alloy is greater than 95%.

11. The stent according to claim 1 wherein the fracture strain A is greater than 40% at room temperature.

12. A stent comprising an iron alloy comprising at least Fe, Cr, N, C, Co and Mn, wherein a cumulative content of Co and Mn is between 3.0 and 20.0% by weight, and
(i) a Cr-eq value for Cr equivalents is greater than 18;
(ii) a Ni-eq value for Ni equivalents is less than 20;
(iii) a PRE value for corrosion resistance is greater than 25;
(iv) Ni-eq>Cr-eq−8; and
(v) 0.25≦C+N≦1.00 and 0.25≦C/N≦1.00.

13. A stent as defined by claim 12 wherein the iron allow further comprises Ni, Co, Mn, Ti, Nb, V, Mo, Si, Al and Cu.

14. A stent as defined by claim 12 wherein components are selected to result in the:
the Cr-eq value being greater than 20;
the Ni-eq value being less than 18;
the PRE value being greater than 28;
the alloy strength Rm being greater than 800 MPa; and, the alloy radial strength being greater than 1.5 bar.

15. A method for making a stent, comprising the step of using an iron alloy to form at least a portion of the stent, the iron alloy having the composition:
Cr greater than 12.0% by weight;
Ni between 0 and 8.0% by weight;
Co between 0 and 20.0% by weight;
Mn between 0 and 20.0% by weight;
N between 0.05 and 1.0% by weight;
C between 0.05 and 0.4% by weight;
Ti between 0 and 3.5% by weight;
Nb between 0 and 3.5% by weight;
V between 0 and 3.5% by weight;
Mo between 0 and 3.5% by weight;
Si between 0 and 3.0% by weight;
Al between 0 and 3.0% by weight; and
Cu between 0 and 3.0% by weight,
wherein a cumulative content of Co and Mn is between 3.0 and 20.0% by weight, and iron and production-related impurities make up a remainder of 100% by weight, and
(i) a Cr-eq value for Cr equivalents that results from the % by weight portions of the stated alloy components represented by formula (1)
Cr-eq=[Cr]+1.5×[Mo]+0.48×[Si]+2.5×[Al]+1.75×[Nb]+2.3×[V] (1)
is greater than 18;
(ii) a Ni-eq value for Ni equivalents that results from the % by weight portions of the stated alloy components represented by formula (2)
Ni-eq=[Ni]+[Co]+30×[C]+18×[N]+0.1×[Mn]−0.01×[Mn]² (2)
is less than 22;
(iii) a PRE value for corrosion resistance that results from the % by weight portions of the stated alloy components represented by formula (3)
PRE=[Cr]+3.3×[Mo]+20×[N] (3)
is greater than 25;
(iv) the limitation represented by formula (4) applies for the Cr-eq and Ni-eq values
Ni-eq>Cr-eq−8 (4);
and
(v) formulas (5) and (6) apply for the limitations on the content of nitrogen and carbon
0.25≦C+N≦1.00 (5),
0.25≦C/N≦1.00 (6)

16. A method as defined by claim 15 wherein the alloy comprises at least 0.05% (by weight) of Ni, Co, Mn, Ti, Nb, V, Mo, Si, Al and Cu.
